(19) United States
(12) Patent Application Publication
(10) Pub. No.: US 2001/0016707 A1
(43) Pub. Date: Aug. 23, 2001

(54) VENTING APPARATUS FOR EYE SURGERY

(76) Inventors: ALEX URICH, MISSION VIEJO, CA (US); MICHAEL CURTIS, LAKE FOREST, CA (US)

Correspondence Address: BEN J. YORKS, ESQ., IRELL & MANELLA LLP, 840 NEWPORT CENTER DRIVE, SUITE 400, NEWPORT BEACH, CA, 92660, US

(21) Appl. No.: 09/351,066

(22) Filed: Jul. 9, 1999

Related U.S. Application Data
(60) Provisional application No. 60/092,676, filed on Jul. 13, 1998.

(51) Int. Cl. A61M 1/00

(52) U.S. Cl. 604/151; 604/67

(57) ABSTRACT

An irrigation/aspiration fluid system that includes a controller which can control an infusion valve and a vent valve. The infusion valve controls the flow of irrigation fluid through an infusion line. The system may also have a pump that creates a vacuum pressure in an aspiration line. By way of example, the infusion line and aspiration line may be coupled to a cornea. The vent valve can control the flow of fluid between the irrigation line and the aspiration line. The controller can control the valves in accordance with a vent routine. In the vent routine the controller switches the pump to an off state and then switches the infusion valve to a closed position. The controller then sequentially opens the vent valve and closes the vent valve to reduce the vacuum pressure in the aspiration line.

### BACKGROUND OF THE INVENTION

1. Field of the Invention

The present invention relates to a medical irrigation/aspiration system used in ophthalmic procedures.

2. Background Information

There are various ophthalmic procedures that require the irrigation and aspiration of fluid from the eye. For example, to replace a natural lens an ultrasonically driven tip is typically inserted into the cornea and manipulated to fragment the lens tissue. Irrigation fluid is introduced into the cornea through an irrigation tube that is inserted into the cornea. The ultrasonically driven tip typically has an aspiration port that is coupled to an aspiration line. The fragmented lens and irrigation fluid is pulled into the aspiration line and directed into a waste container.

FIG. 1 shows an irrigation/aspiration system of the prior art. The system includes an infusion line 1 that is connected to a bottle of irrigation fluid 2. The infusion line 1 can be inserted into a cornea 3. The infusion line 1 has an infusion valve 4 that can be turned off to terminate the flow of irrigation fluid into the cornea when a procedure is completed.

The system also includes an aspiration line 5 that is coupled to a pump 6 and is inserted into the cornea 3. The pump 6 creates a vacuum pressure within the line 5 to pull the irrigation fluid and fragmented tissue from the cornea 3.

When a procedure is completed the surgeon turns the pump 6 off and switches the infusion valve 4 to a closed position to terminate the flow of irrigation fluid into the cornea 3. When the system is in this state the aspiration line 5 still has a vacuum pressure that may cause the eye to collapse. To prevent corneal collapse the system also includes a vent line 7 and a vent valve 8 that can be opened to eliminate the vacuum in the aspiration line 5.

It has been found that during the vent cycle fluid will also flow from the cornea 3 to the fluid bottle 2. This flow of fluid can create a “bounce” in the cornea 3 that may damage parts of the eye. To prevent this back flow of fluid a one-check valve 9 can be installed into the irrigation line 1. The valve 9 may fail without warning. Any failure of the check valve could produce unwanted effects to the eye. It would be desirable to provide an irrigation/aspiration system that prevents corneal bounce without a check valve.

### SUMMARY OF THE INVENTION

One embodiment of the present invention is an irrigation/aspiration fluid system that includes a controller which can control an infusion valve and a vent valve. The infusion valve controls the flow of irrigation fluid through an infusion line. The system may also have a pump that creates a vacuum pressure in an aspiration line. By way of example, the infusion line and aspiration line may be coupled to a cornea. The vent valve can control the flow of fluid between the irrigation line and the aspiration line. The controller can control the valves in accordance with a vent routine. In the vent routine the controller switches the pump to an off state and then switches the infusion valve to a closed position. The controller then sequentially opens the vent valve and closes the vent valve to reduce the vacuum pressure in the aspiration line.

### BRIEF DESCRIPTION OF THE DRAWINGS

FIG. 1 is a schematic of an irrigation/aspiration system of the prior art;

FIG. 2 is a schematic of an irrigation/aspiration system of the present invention.

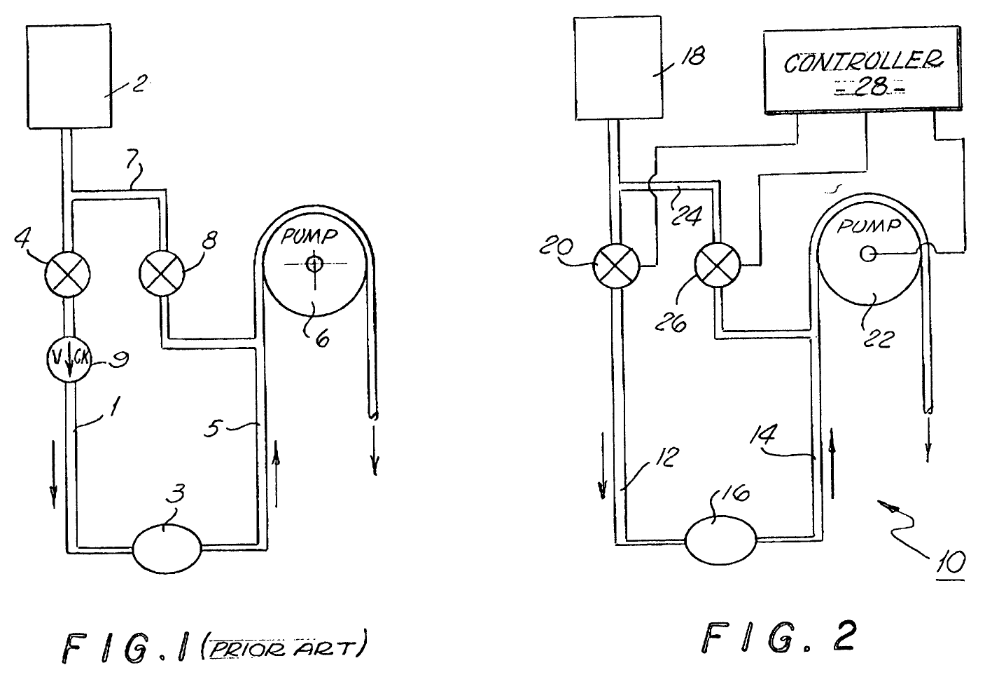

### DETAILED DESCRIPTION

Referring to the drawings more particularly by reference numbers, FIG. 2 shows an irrigation/aspiration system 10 of the present invention. The system 10 may include an infusion line 12 and an aspiration line 14 that can be coupled to a cornea 16. Although a cornea 16 is depicted and described, it is to be understood that the system 10 can be used for medical procedures on other parts of the anatomy.

The infusion line 12 may be connected to a source of fluid 18. The source of fluid 18 may be a bottle filled with irrigation fluid that is elevated above the cornea 16. The elevated bottle allows the irrigation fluid to flow through the line 12 and into the cornea under the force of gravity. The flow of fluid through the line 12 can be controlled by an infusion valve 20. The infusion valve 20 can be switched between an open position and a closed position.

The aspiration line 14 may be coupled to a vacuum pump 22. The vacuum pump 22 may be a non-invasive peristaltic pump that creates a vacuum pressure in the aspiration line 14 as is known in the art. The pump 22 can be switched between an on state and an off state.

The system 10 may have a vent line 24 that is connected to the infusion 12 and aspiration 14 lines. The flow of fluid through the vent line 24 may be controlled by a vent valve 26. The vent valve 26 can be switched between an open position and a closed position.

The system 10 may further include an electronic controller 28 that is connected to the infusion valve 20, the vent valve 26 and the pump 22. The electronic controller 28 may contain a processor, memory, driver circuits, etc. that can control and switch the pump 22 and valves 20 and 26 in accordance with a software program such as a vent routine. The controller 28 may enter the vent routine when the surgical procedure is completed and the irrigation/aspiration system 10 is to be detached from the cornea 16.

In the vent routine the controller 28 may initially switch the pump 22 to the off state. After a time delay the controller 28 may switch the infusion valve 20 to the closed position to terminate flow through the infusion line. By way of example, the time delay may be approximately 100 milliseconds.

After the infusion valve 20 is closed the controller 28 may switch the vent valve 26 to an open position to allow irrigation fluid to flow from the infusion line 12 to the aspiration line 14. There may be a time delay of approximately 100 milliseconds between the closing of the infusion valve 20 and the opening of the vent valve 26.

The controller 28 switches the vent valve 26 to the closed position when the aspiration line pressure is equal to, or approximately equal to, the infusion line pressure. The system 10 may have a pressure sensor (not shown) in the aspiration line 14 to provide feedback to the controller 28. Coupling the aspiration line 14 to the infusion line 12 while the infusion valve 20 is closed prevents a reverse flow of fluid from the cornea 16 to the bottle 18 and eliminates a cornea bounce during the vent cycle.

The controller 28 may further open and close the infusion valve 20 for a certain time duration after the vent valve 26 is closed to further equalize the pressure within the cornea 16 and fluid lines 12 and 16. The time duration may be approximately 500 milliseconds.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. An irrigation/aspiration system, comprising:
source of fluid;
an infusion line connected to said source of fluid;
an infusion valve that can control a flow of fluid through said infusion line and can be switched between an open position and a closed position;
a pump that can be switched between an on state and an off state;
an aspiration line connected to said pump;
a vent line connected to said infusion line and said aspiration line;
a vent valve that can control a flow of fluid through said vent line and can be switched between an open position and a closed position; and,
a controller that is connected to said pump, said infusion valve and said vent valve, said controller can control said infusion and vent valves and said pump in accordance with a vent routine wherein said pump is switched to the off state and said infusion valve is switched to the closed position, said vent valve is then switched to said open position, said vent and infusion valves are subsequently switched to said closed position.

2. The system of claim 1, wherein said controller waits a first predetermined time interval between switching said pump to the off state and switching said infusion valve to the closed position.

3. The system of claim 1, wherein said controller waits a first predetermined time interval between switching said infusion valve to the closed position and switching said vent valve to the open position.

4. The system of claim 2, wherein said controller waits a second predetermined time interval between switching said infusion valve to the closed position and switching said vent valve to the open position.

5. The system of claim 4, wherein said first and second predetermined time periods are each approximately 100 milliseconds.

6. The system of claim 1, wherein said controller opens said infusion valve and then closes said infusion valve after said vent valve is closed.

7. The system of claim 6, wherein said controller waits a predetermined time period between opening and closing said infusion valve.

8. A method for venting an irrigation/aspiration system, comprising:
switching a pump to an off state;
switching an infusion valve to a closed position;
switching a vent valve to an open position;
switching the vent valve to a closed position; and, switching the infusion valve to a closed position.

9. The method of claim 8, further comprising switching the infusion valve to the open position and then switching the infusion valve to the closed position.

10. The method of claim 8, further comprising waiting a predetermined time interval between switching the pump to the off state and switching the infusion valve to the closed position.

11. The method of claim 8, further comprising waiting a predetermined time interval between switching the infusion valve between the off position and switching the vent valve to the open position.

12. The method of claim 9, further comprising waiting a predetermined time interval between the opening and closing of the infusion valve.
